(19) United States
(12) Patent Application Publication
(10) Pub. No.: US 2025/0359848 A1
(43) Pub. Date: Nov. 27, 2025

(54) MEDICAL IMAGING PROBE AND MEDICAL IMAGING SYSTEM

(71) Applicant: GE Precision Healthcare LLC, Waukesha, WI (US)

(72) Inventors: Xin Ge, Wuxi (CN); Qifei Leng, Wuxi (CN); Gengjie Du, Wuxi (CN)

(21) Appl. No.: 19/216,391

(22) Filed: May 22, 2025

(30) Foreign Application Priority Data
May 23, 2024 (CN) 202410649651.7

(51) Int. Cl. A61B 8/00

(52) U.S. Cl. CPC A61B 8/4461; A61B 2560/0462; A61B 2562/0219; A61B 2562/168

(57) ABSTRACT

A medical imaging probe including a housing; a scanning head, movably disposed within the housing, the scanning head including: a movable transducer module, a transducer array and a backing, there being a gap between the transducer array and a contact surface of the housing, a base coupled to the transducer module and disposed opposite the transducer array, an electromagnet disposed on one of the transducer module and the base, and a magnetic attraction material disposed on the other of the transducer module and the base; and a sensor, the sensor being configured to sense an acceleration state of the medical imaging probe exceeding a threshold. In response to the sensor sensing the acceleration state, the electromagnet is energized to attract the magnetic attraction material, to cause the transducer module to be proximate to the base and the gap between the transducer array and the contact surface to increase.

### CROSS-REFERENCE TO RELATED APPLICATION

This application claim priority to Chinese Patent Application No. 202410649651.7, which was file on May 23, 2024 at the Chinese Patent Office. The entire contents of the above-listed application are incorporated by reference herein in their entirety.

### TECHNICAL FIELD

The present application relates to the field of medical imaging, and more specifically, to a medical imaging probe and a medical imaging system.

### BACKGROUND

In general, a medical imaging probe may be used to image a subject under examination, to perform scanning and examination. The manner in which a medical imaging device generates an image depends on a particular technology.

For example, ultrasound imaging uses real-time, non-destructive high-frequency acoustic waves to produce ultrasound images, e.g., ultrasound images of organs, tissues, and subjects (e.g., fetuses) within a human body. The images produced or generated by the medical imaging device may be two-dimensional (2D), three-dimensional (3D) and/or four-dimensional (4D) images (essentially real-time/continuous 3D images). During medical imaging, an imaging dataset (including, for example, a volumetric imaging dataset of a 3D/4D imaging device) is acquired, and corresponding images are generated and rendered in real time using the imaging dataset.

A medical imaging probe typically includes structurally compact components. During use of the medical imaging probe, accidental events such as collisions and drops may occur, and impact therefrom may cause damage to internal components of the probe. This may lead to reliability issues and may lead to increased probe service and exchange costs.

### SUMMARY OF THE INVENTION

The objective of the present invention is intended to overcome the above-mentioned and/or other problems in the prior art. According to the present invention, a medical imaging probe and a medical imaging system are provided, which can sense an acceleration state of the medical imaging probe exceeding a threshold, and in response to sensing the acceleration state, allow relative movement of components within the medical imaging probe, so as to protect the components within the medical imaging probe.

According to a first aspect of the present invention, provided is a medical imaging probe comprising: a housing, a front end of the housing being sealed with a contact surface; a scanning head, the scanning head being movably disposed within the housing, wherein the scanning head comprises: a movable transducer module, the movable transducer module comprising a transducer array and a backing, the transducer array being opposite the contact surface, and there being a gap between the transducer array and the contact surface, a base, the base being coupled to the transducer module and disposed opposite the transducer array, an electromagnet, the electromagnet being disposed on one of the transducer module and the base, and a magnetic attraction material, the magnet attraction material being disposed on the other of the transducer module and the base; and a sensor, the sensor being configured to sense a acceleration state of the medical imaging probe exceeding a threshold. The electromagnet and the magnetic attraction material are positioned in such a way that, in response to the acceleration sensor sensing the acceleration state, the electromagnet is energized to attract the magnetic adsorbent material, causing the transducer module to be proximate to the base, and the gap between the transducer array and the contact surface to increase.

In one embodiment, the medical imaging probe further comprises a resilient member, the resilient member being coupled between the transducer module and the base, and configured to be compressible to allow relative movement between the base and the transducer module.

In one embodiment, when the electromagnet is de-energized, the resilient member resets the transducer module to restore the gap.

In one embodiment, the resilient member comprises at least one spring coupled between the transducer module and the base.

In one embodiment, the electromagnet is disposed on the base, and the magnetic attraction material is disposed on the transducer module.

In one embodiment, the magnetic attraction material is disposed on a bottom surface of the transducer module opposite the transducer array.

In one embodiment, when the electromagnet is energized to attract the magnetic attraction material, the transducer module is moved towards the base.

In one embodiment, the electromagnet can be energized only during non-operation periods of the transducer module.

In one embodiment, the housing comprises a wet chamber filled with an acoustic liquid, and a dry chamber in which the transducer module is disposed, wherein, when the electromagnet is energized to attract the magnetic attraction material, the acoustic liquid is more filled into the gap between the transducer module and the contact surface.

In one embodiment, the spacing between the transducer module and the base is configured to gradually increase from inside to outside in at least one radial direction.

In one embodiment, the base comprises a shaft around which the scanning head can be rotated.

In one embodiment, the medical imaging probe further comprises a driver, the driver driving the scanning head to rotate around the shaft.

According to a second aspect of the present invention, provided is a medical imaging system, which comprises the medical imaging probe of any one of the foregoing.

### BRIEF DESCRIPTION OF THE DRAWINGS

The present invention can be better understood by means of the description of the exemplary embodiments of the present invention in conjunction with the drawings, in which:

FIG. 1 is a block diagram illustrating an exemplary medical imaging arrangement.

FIG. 2 is a block diagram illustrating an exemplary ultrasound system.

FIG. 3 is a schematic diagram of an exemplary ultrasound probe according to one embodiment.

FIG. 4 is a partial schematic diagram of the exemplary ultrasound probe according to one embodiment, excluding a housing.

FIG. 5 is a partial perspective view of a part of an exemplary ultrasound probe according to one embodiment of the present invention.

FIG. 6 is a schematic diagram of a scanning head 501 in an attracting state according to one embodiment of the present invention.

FIG. 7 is a schematic diagram of a medical imaging system according to one embodiment of the present invention.

In the accompanying drawings, similar components and/or features may have the same numerical reference signs. Further, components of the same type may be distinguished by letters following the reference sign, and the letters may be used for distinguishing between similar components and/or features. If only a first numerical reference sign is used in the specification, the description is applicable to any similar component and/or feature having the same first numerical reference sign irrespective of the subscript of the letter.

### DETAILED DESCRIPTION

Specific implementations of the present invention will be described below. It should be noted that in the specific description of said implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

References in the specification to “an embodiment,” “embodiment,” “exemplary embodiment,” and so on indicate that the embodiment described may include a specific feature, structure, or characteristic, but the specific feature, structure, or characteristic is not necessarily included in every embodiment. Besides, such phrases do not necessarily refer to the same embodiment. Further, when a specific feature, structure, or characteristic is described in connection with an embodiment, it is believed that affecting such feature, structure, or characteristic in connection with other embodiments (whether or not explicitly described) is within the knowledge of those skilled in the art.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

Unless defined otherwise, technical terms or scientific terms used in the claims and description should have the usual meanings that are understood by those of ordinary skill in the technical field to which the present invention belongs. The terms “include” or “comprise” and similar words indicate that an element or object preceding the terms “include” or “comprise” encompasses elements or objects and equivalent elements thereof listed after the terms “include” or “comprise”, and do not exclude other elements or objects.

In addition, as used herein, the term “image” broadly refers to both a viewable image and data representing a viewable image. However, many embodiments generate (or are configured to generate) at least one viewable image. In addition, as used herein, as used in the ultrasound imaging environment, the phrase “image” is used to refer to an ultrasound mode, such as a B mode (2D mode), an M mode, a three-dimensional (3D) mode, a CF mode, PW Doppler, CW Doppler, MGD and/or a B sub-mode and/or a CF sub-mode, such as shear wave elasticity imaging (SWEI), TVI, Angio, B-flow, BMI and BMI_Angio, and in some cases, MM, CM and TVD, where “image” and/or “plane” includes a single beam or a plurality of beams.

Furthermore, as used herein, the term “processor” or “processing unit” refers to any type of processing unit that can perform desired computations required by various implementations, such as a single-core or multi-core CPU, accelerated processing unit (APU), graphics board, DSP, FPGA, ASIC, or a combination thereof.

It should be noted that various implementations of generating or forming images described herein may include processing for forming images, which includes beamforming in some implementations and excludes beamforming in other implementations. For example, images may be formed without performing beamforming, such as by multiplying a matrix of demodulated data by a coefficient matrix, such that the product is an image, and where this process does not form any “beams”. Furthermore, formation of images may be performed using channel combinations (e.g., synthetic aperture techniques) potentially derived from more than one transmit event.

In various implementations, the processing for forming images is executed in software, firmware, hardware, or a combination thereof. The processing may include the use of beamforming.

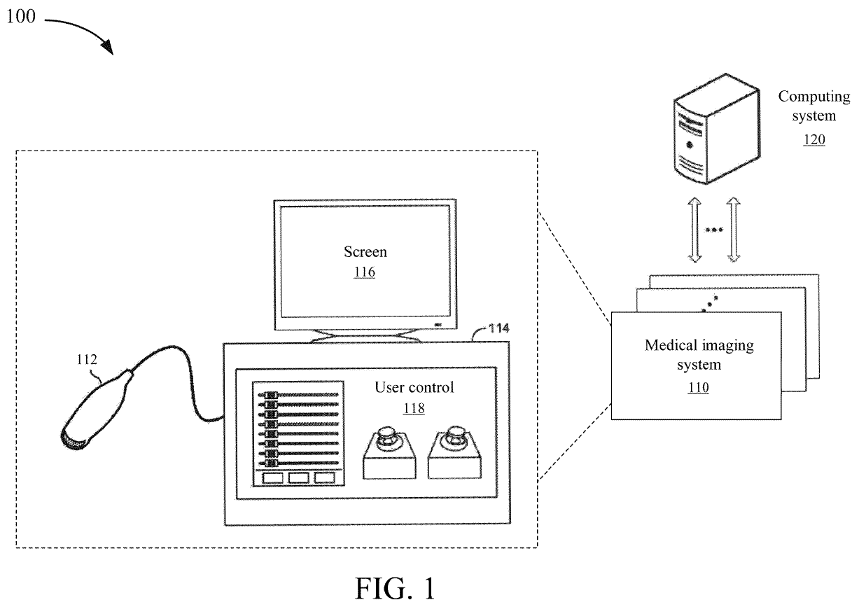

FIG. 1 is a block diagram illustrating an exemplary medical imaging arrangement. FIG. 1 shows an exemplary medical imaging arrangement 100, including one or more medical imaging systems 110 and one or more computing systems 120. The medical imaging arrangement 100 (including various elements) may be configured to support medical imaging and solutions associated therewith.

The medical imaging system 110 includes suitable hardware, software, or a combination thereof to support medical imaging (i.e., enabling acquisition of data for generating and/or rendering images during a medical imaging examination). An example of medical imaging may be ultrasound imaging. This may require capturing a specific type of data in a specific manner, and the data can then be used to generate data for an image. For example, the medical imaging system 110 may be an ultrasound imaging system configured to generate and/or render ultrasound images.

As shown in FIG. 1, the medical imaging system 110 may include a scanner device 112 and a display/control unit 114, and the scanner device may be portable and movable. The scanner device 112 may be configured to generate and/or capture specific types of imaging signals (and/or data corresponding thereto) by, for example, moving over a patient's body (or a portion thereof), and may include suitable circuits for performing and/or supporting such functions. The scanner device 112 may be an ultrasound probe, such as a 4D ultrasound probe. In this scenario, the scanner device 112 may emit an ultrasound signal and capture an echo ultrasound image.

The display/control unit 114 may be configured to display images (e.g., via a screen 116). In some cases, the display/control unit 114 may also be configured to at least partially generate the displayed images. In addition, the display/control unit 114 may further support user input/output. For example, in addition to images, the display/control unit 114 may further provide (e.g., via the screen 116) user feedback (e.g., information related to the system, the functions and settings thereof, etc.). The display/control unit 114 may further support user input (e.g., via user controls 118) to, for example, allow control of medical imaging. User input can involve controlling the display of images, selecting settings, specifying user preferences, requesting feedback, etc.

In some specific implementations, the medical imaging arrangement 100 may further include additional and dedicated computing resources, such as one or more computing systems 120. In this regard, each computing system 120 may include circuits, interfaces, logic, and/or code suitable for processing, storing, and/or communicating data. The computing system 120 may be a specialized device configured for use specifically in conjunction with medical imaging, or it may be a general-purpose computing system (e.g., a personal computer, server, etc.) that is set up and/or configured to perform the operations described below with respect to computing system 120. The computing system 120 may be configured to support the operation of the medical imaging system 110, as described below. In this regard, various functions and/or operations can be offloaded from the imaging system, which may simplify and/or centralize certain aspects of processing to reduce costs, for example, by eliminating the need to add processing resources to the imaging system.

The computing system 120 may be set up and/or arranged for use in different ways. For example, in some specific implementations, a single computing system 120 may be used; and in other specific implementations, a plurality of computing systems 120 are configured to work together (for example, configured based on distributed processing), or individually. Each of the computing systems 120 is configured to process a specific aspect and/or function, and/or to process data only for a specific medical imaging system 110. In addition, in some specific implementations, the computing system 120 may be local (for example, co-located with one or more medical imaging systems 110, such as within the same facility and/or the same local network); and in other specific embodiments, the computing system 120 may be remote, and thus accessible only by means of a remote connection (for example, by means of the Internet or other available remote access technologies). In particular specific implementations, the computing system 120 may be configured in a cloud-based manner and may be accessed and/or used in a substantially similar manner to accessing and using other cloud-based systems.

Once data is generated and/or configured in the computing system 120, the data can be copied and/or loaded into the medical imaging system 110. This can be done in different ways. For example, the data may be loaded via a directed connection or link between the medical imaging system 110 and the computing system 120. In this regard, communication between the different elements of the medical imaging arrangement 100 can be performed using available wired and/or wireless connections, and/or according to any suitable communication (and/or networking) standards or protocols. Alternatively or additionally, the data may be indirectly loaded into the medical imaging system 110. For example, the data may be stored in a suitable machine-readable medium (for example, a flash memory card) and then loaded into the medical imaging system 110 using the machine-readable medium (on-site, for example, by a user of the system (such as an imaging clinician) or authorized personnel); alternatively, the data may be downloaded to a locally communicative electronic device (for example, a laptop) and then the electronic device used on-site (for example, by a user of the system or authorized personnel) to upload the data to the medical imaging system 110 by means of a direct connection (for example, a USB connector).

In operation, the medical imaging system 110 may be used to generate and present (for example, render or display) images during a medical examination, and/or used in conjunction therewith to support user input/output. The images can be 2D, 3D, and/or 4D images. The particular operations or functions performed in the medical imaging system 110 to facilitate the generation and/or presentation of images depend on the type of system (for example, the means used to obtain and/or generate the data corresponding to the images). For example, in ultrasound imaging, the data is based on the emitted and echo ultrasound signals.

In various specific implementations according to the present disclosure, the medical imaging system and/or architecture (e.g., the medical imaging system 110 and/or the medical imaging apparatus 100 as a whole) may be configured to support a medical imaging probe being implemented and utilized.

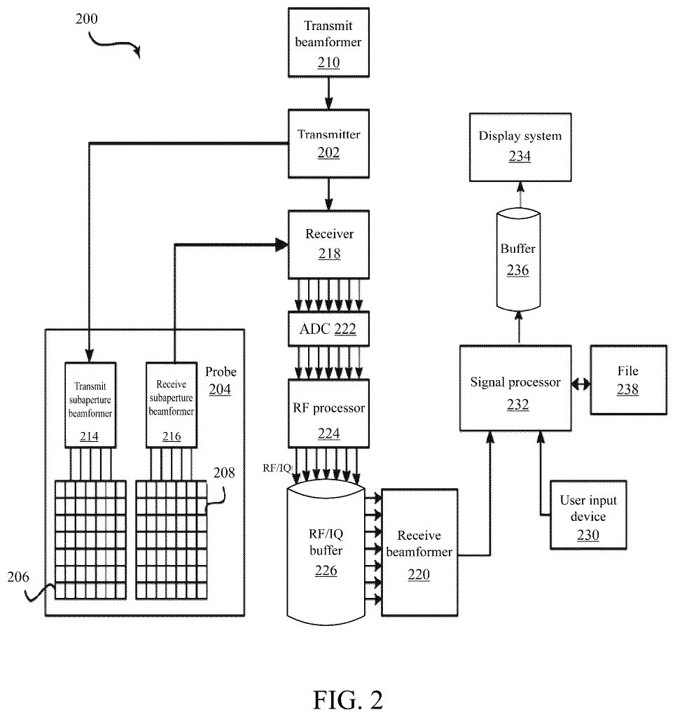

FIG. 2 is a block diagram illustrating an exemplary ultrasound system 200. The ultrasound system 200 includes a transmitter 202, an ultrasound probe 204, a transmit beamformer 210, a receiver 218, a receive beamformer 220, an A/D converter 222, an RF processor 224, an RF/IQ buffer 226, a user input device 230, a signal processor 232, an image buffer 236, a display system 234, and a file 238.

The transmitter 202 may include suitable logic, circuitry, interfaces, and/or codes, which may be operated to drive the ultrasound probe 204. The ultrasound probe 204 may be, for example, an E4D probe (electronic 4D probe) or a mechanical rotating probe. The E4D probe may be a linear E4D probe, a curved E4D probe, or a sector E4D probe. The mechanical rotating probe may be a linear mechanical rotating probe, a curved mechanical rotating probe, or a sector mechanical rotating probe. The ultrasound probe 204 may be configured to acquire both 2D B-mode data and 2D color blood flow data, or to acquire both 2D B-mode data and another ultrasound mode that detects a blood flow velocity in the direction of the vascular axis. The ultrasound probe 204 may include a two-dimensional (2D) array of piezoelectric elements. The ultrasound probe 204 may include a set of transmitting transducer elements 206 and a set of receiving transducer elements 208 that typically form the same element. In some implementations, the ultrasound probe 204 may be operated to acquire ultrasound image data covering at least most of an anatomical structure (such as a heart, a blood vessel, or any suitable anatomical structure).

The transmitting beamformer 210 may include suitable logic, circuitry, interfaces, and/or code that may be operated to control the transmitter 202, and the transmitter 202 drives the set of transmitting transducer elements 206 by means of a transmit subaperture beamformer 214 to transmit ultrasound emission signals into a region of interest (e.g., a person, animal, subsurface cavity, physical structure, etc.). The emitted ultrasound signal can be backscattered from structures in the subject of interest (e.g., blood cells or tissue) to produce echoes. The echo is received by the receiving transducer element 208.

The set of receiving transducer elements 208 in the ultrasound probe 204 can be configured to convert the received echo to an analog signal, perform subaperture beamforming by means of a receive subaperture beamformer 216, and then transmit the analog signal to the receiver 218. The receiver 218 may include suitable logic, circuitry, interfaces, and/or code that may be operated to receive signals from the receiving subaperture beamformer 216. The analog signal can be transferred to one or more of a plurality of A/D converters 222.

The plurality of A/D converters 222 may include suitable logic, circuitry, interfaces, and/or code that may be operated to convert the analog signal from the receiver 218 to a corresponding digital signal. The plurality of A/D converters 222 are disposed between the receiver 218 and the RF processor 224. Nevertheless, the present disclosure is not limited in this regard. Thus, in some implementations, the plurality of A/D converters 222 may be integrated within the receiver 218.

The RF processor 224 may include suitable logic, circuitry, interfaces, and/or code that may be operated to demodulate the digital signals output by the plurality of A/D converters 222. According to one implementation, the RF processor 224 may include a complex demodulator (not shown) that can be used to demodulate the digital signal to form an I/Q data pair representing the corresponding echo signal. The RF or I/Q signal data can then be transferred to the RF/IQ buffer 226. The RF/IQ buffer 226 may include suitable logic, circuitry, interfaces, and/or code that may be operated to provide temporary storage of RF or I/Q signal data generated by the RF processor 224.

The receive beamformer 220 may include suitable logic, circuitry, interfaces, and/or code that may be operated to perform digital beamforming processing to, for example, sum delayed channel signals received via the RF/IQ buffer 226 from the RF processor 224 and output beam-summed signals. The resulting processed information may be the beam-summed signals outputted from the receive beamformer 220 and transmitted to the signal processor 232. According to some implementations, the receiver 218, the plurality of A/D converters 222, the RF processor 224, and the beamformer 220 may be integrated into a single beamformer, and said single beamformer may be digital. In various embodiments, the ultrasound system 200 includes a plurality of receiving beamformers 220.

The user input device 230 can be used to input patient data, scan parameters, and settings, select protocols and/or templates, etc. In an illustrative implementation, the user input device 230 may be operated to configure, manage, and/or control the operation of one or more components and/or modules in the ultrasound system 200. In this regard, the user input device 230 can be used to configure, manage, and/or control the operation of the transmitter 202, the ultrasound probe 204, the transmit beamformer 210, the receiver 218, the receive beamformer 220, the RF processor 224, the RF/IQ buffer 226, the user input device 230, the signal processor 232, the image buffer 236, the display system 234, and/or the file 238. The user input devices 230 may include buttons, rotary encoders, touch screens, motion tracking, voice recognition, mouse devices, keyboards, cameras, and/or any other devices capable of receiving user commands. In some implementations, for example, one or more of the user input devices 230 may be integrated into other components (such as the display system 234 or the ultrasound probe 204). For example, the user input device 230 may include a touch screen display.

The signal processor 232 may include suitable logic, circuitry, interfaces, and/or code that may be operated to process the ultrasound scan data (i.e., the summed IQ signal) to generate an ultrasound image for presentation on the display system 234. The signal processor 232 may be operated to perform one or more processing operations based on a plurality of selectable ultrasound modalities on the acquired ultrasound scan data. In an illustrative implementation, the signal processor 232 can be used to execute display processing and/or control processing, etc. As the echo signal is received, the acquired ultrasound scan data can be processed in real-time during the scan session. Additionally or alternatively, the ultrasound scan data may be temporarily stored in the RF/IQ buffer 226 during the scan session and processed in a less real-time manner during online or offline operation. In various implementations, the processed image data may be presented at the display system 234 and/or may be stored in the file 238. The file 238 can be a local file, a picture archiving and communication system (PACS), or any suitable device for storing images and related information.

The signal processor 232 may be one or more central processing units, microprocessors, microcontrollers, etc. For example, the signal processor 232 may be an integrated component, or may be distributed in various locations. In an illustrative implementation, the signal processor 232 may be able to receive input information from the user input device 230 and/or file 238, generate outputs that may be shown by the display system 234, manipulate the outputs, etc., in response to the input information from the user input device 230. The signal processor 232 may be capable of executing, for example, any of the methods and/or instruction sets discussed herein according to various implementations.

The ultrasound system 200 may be configured to continuously acquire ultrasound scan data at a frame rate suitable for the imaging situation under consideration. Typical frame rates are in the range of 20 to 120, but can be lower or higher. The acquired ultrasound scan data can be shown on the display system 234 at the same display rate as the frame rate, or slower or faster than the frame rate. The image buffer 236 is included to store for processing frames of the acquired ultrasound scan data that are not scheduled for immediate display. Preferably, the image buffer 236 has sufficient capacity to store frames of ultrasound scan data for at least a few minutes. Frames of ultrasound scan data are stored in such a way that they can be easily retrieved therefrom according to their acquisition sequence or time. The image buffer 236 may be embodied in any known data storage medium.

The display system 234 may be any device capable of communicating visual information to users. For example, the display system 234 may include a liquid crystal display, a light emitting diode display, and/or any one or more suitable displays. The display system 234 may be operated to present ultrasound images and/or any suitable information.

The file 238 may be one or more computer-readable memories integrated with and/or communicatively coupled (e.g., via a network) to the ultrasound system 200, such as a Picture Archiving and Communication System (PACS), a server, a hard disk, a floppy disk, a CD, a CD-ROM, a DVD, a compact memory, a flash memory, a random access memory, a read only memory, an electrically erasable and programmable read only memory, and/or any suitable memory. The file 238 may include, for example, a database, a library, an information set, or other memory accessed by the signal processor 232 and/or incorporated into the signal processor 232. For example, the file 238 can temporarily or permanently store data. The file 238 may be capable of storing medical image data, data generated by the signal processor 232, and/or instructions readable by the signal processor 132, etc.

Components of the ultrasound system 200 may be implemented in software, hardware, firmware, etc. Various components of the ultrasound system 200 may be communicatively connected. The components of the ultrasound system 200 may be implemented individually and/or integrated in various forms. For example, the display system 234 and the user input device 230 may be integrated as a touch screen display.

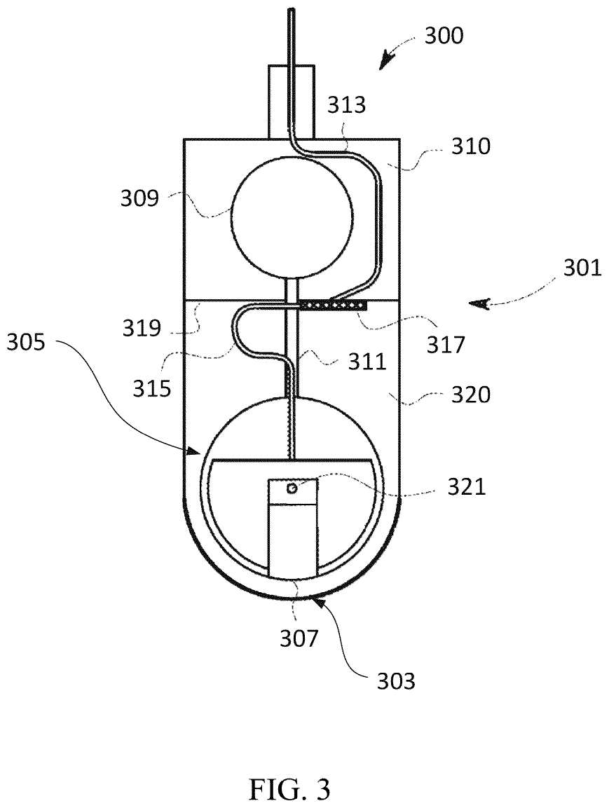

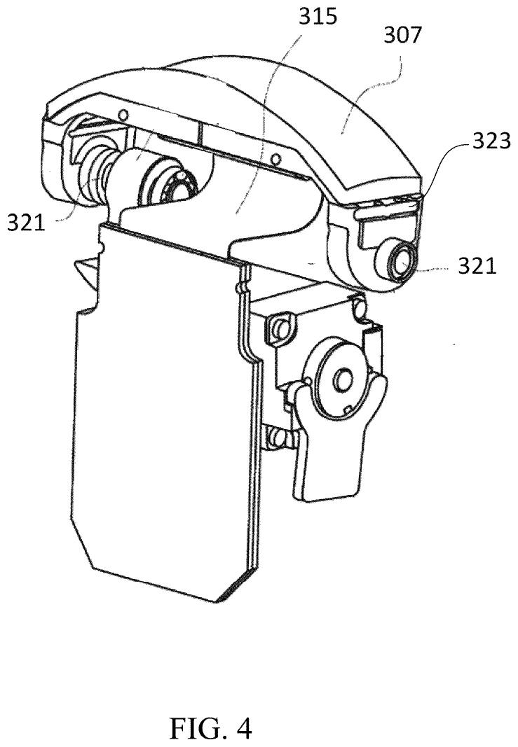

As previously described, the ultrasound probe 206 may be a mechanical ultrasound probe. FIG. 3 is a schematic diagram of an exemplary ultrasound probe according to one embodiment. In one embodiment, the ultrasound probe 300 includes a housing 301, and a front end of the housing 301 is sealed with a contact surface 303. A scanning head 305 is movably disposed within the housing 301. FIG. 4 is a partial schematic diagram of the exemplary ultrasound probe according to one embodiment, excluding the housing. The structure of the exemplary ultrasound probe is described below with reference to FIGS. 3 and 4.

The housing 301 may have a first chamber 310 (e.g., a dry chamber) and a second chamber 320 (e.g., a wet chamber). The first chamber 310 and the second chamber 320 may be formed as a single unit (e.g., an integral construction) or may be formed as separate units that are connected together. In an exemplary embodiment, the first chamber 310 is a dry chamber or an air chamber, in which drive components for mechanically controlling the transducer array 307 and communication components for electrically controlling the transducer array 307 are provided. The drive components generally include a motor 309 (e.g., a stepper motor) and a gear arrangement 311. The communication components generally include a system cable 313, and the system cable 313 is connected to a flexible PCB 315, so as to communicate with a host system to drive elements of the transducer array 307 (e.g., to selectively activate the elements of the transducer array 307).

In some embodiments, only a single dry chamber is provided. Furthermore, although the drive components and the communication components are described herein as having specific assembly parts, these components are not limited thereto. For example, the drive components may have different gear arrangements and the communication components may have different connecting members or transmission lines.

In this exemplary embodiment, the second chamber 320 is a wet chamber, e.g., a chamber having an acoustic liquid therein, in which transducer drive components for moving (e.g., rotating) the transducer array 307 and transducer control components for selectively driving the elements (e.g., piezoelectric ceramics) of the transducer array 307 are provided.

The transducer drive components generally include a transducer shaft 321 connected to the scanning head 305 (e.g., coupled to the scanning head 305) and extending within a drive shaft opening formed within the scanning head 305. A connector support member 323 is also coupled within the scanning head 305, so as to support the flexible PCB 315 connected to the transducer array 307. The scanning head 305 generally defines a transducer carrier or transducer bridge, so that when the transducer shaft 321 moves (e.g., rotates) to move the scanning head 305, movement of the transducer array 307 mounted thereto is also provided. It should be noted that the flexible PCB 315 is coupled between the connector support member 323 and the transducer array 307, and is electrically connected to the transducer array 307.

The transducer control components generally include a connection member 317, the connection member 317 being used to interconnect the system cable 313 with the flexible PCB 315 (e.g., four scanning head flexible printed circuit boards), and the flexible PCB 315 having one or more communication lines for communication therebetween. In one exemplary embodiment, the connection member 317 is formed from one or more rigid printed circuit boards, which interconnect the system cable 313 with the flexible PCB 315 via a sealing member 319 that provides a liquid seal between the first chamber 310 and the second chamber 320.

It should also be noted that although the transducer drive components and transducer control components are described herein as having particular assembly parts, these components are not limited thereto. For example, the transducer drive components may have different shaft arrangements, and the transducer control components may have different control circuits or transmission lines. It should also be noted that additional or different assembly parts may be provided to connect the probe 300 as needed or desired, and/or based on the particular type and application of the probe 300. It should also be noted that the transducer array 307 may be configured for operation in different modes, such as 1D, 1.25D, 1.5D, 1.75D, 2D, 3D, and 4D operating modes.

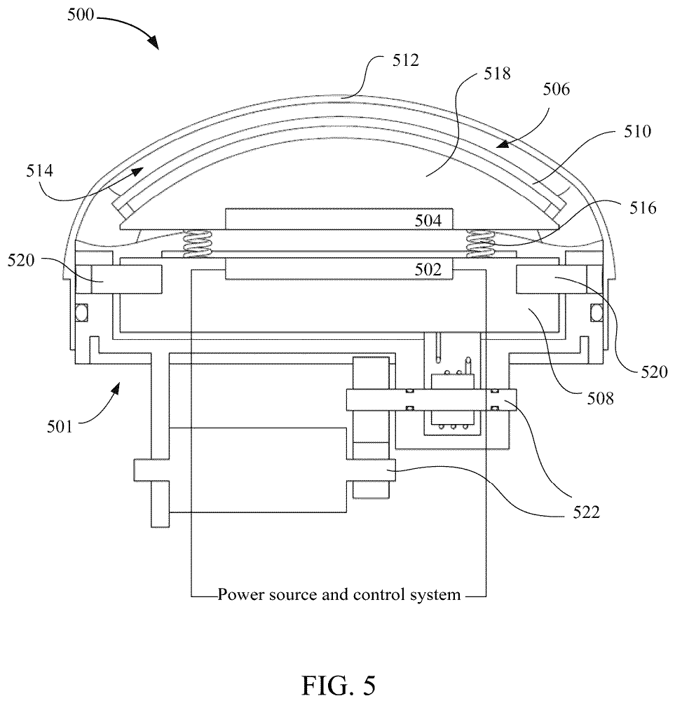

FIG. 5 is a partial perspective view of a part of an ultrasound probe 500 according to one embodiment of the present invention.

A medical imaging probe (e.g., an ultrasound probe) may include fragile components, e.g., a transducer array. These components may be located near a contact surface of the probe, and may be susceptible to damage or breakage, particularly when, for example, accidental events such as drops and impacts occur. Components inside the medical imaging probe may be damaged due to the impacts. This may lead to reliability issues, and may lead to increased probe service and exchange costs. Therefore, there is a need for a solution that can reduce the risk of damage to components inside the medical imaging probe. To this end, the ultrasound probe 500 may include a sensor (not shown), the sensor being configured to sense an acceleration state of the ultrasound probe. The sensor may be disposed at any suitable location within the housing. The ultrasound probe 500 may have a housing, a front end of the housing being sealed with a contact surface 512. The ultrasound probe 500 may include a scanning head 501, the scanning head 501 being movably disposed within the housing, and the scanning head including a movable transducer module 506, including a transducer array 510 and a backing 518. The ultrasound probe 500 may include an electromagnet 502 and a magnetic attraction material 504, where the electromagnet 502 may be disposed on any one of the transducer module 506 and a base 508, and the magnetic attraction material 504 is disposed on the other of the transducer module 506 and the base 508. As one example, in FIG. 5, the electromagnet 502 is shown as being disposed on the base 508 and the magnetic attraction material 504 is shown as being disposed on the transducer module 506. Preferably, the magnetic attraction material 504 is disposed on a bottom surface of the transducer module 506 opposite the transducer array 510.

The electromagnet and the magnetic attraction material are positioned in such a way that, in response to the sensor detecting that the ultrasound probe 500 is in an acceleration state, the electromagnet 502 is energized to attraction the magnetic attraction material 504, causing the transducer module 506 to be proximate to the base 508, and a gap 514 between the transducer array 510 and the contact surface 512 to thereby increase.

Such a configuration can ensure that, in the event of an accidental fall or the like of the ultrasound probe 500, the fragile transducer module 506 is as far away from the housing as possible, to avoid impacts.

In some embodiments, operation of the electromagnet 502 is controlled by a control system, and energized by a power source of the electromagnet 502. Optionally, the power source of the electromagnet 502 may be disposed inside the ultrasound probe 500, or in cases where the ultrasound probe 500 has an external power source to support the operation of the ultrasound probe 500, the electromagnet 502 may also be powered by the external power source of the ultrasound probe 500. Additionally, the type of the sensor described above may be any in the art, for example, a sensor that senses a change in the motion state of an object, such as an acceleration sensor.

Furthermore, when the ultrasound probe 500 is in a normal state, the electromagnet 502 may be de-energized, causing the transducer module 506 to be reset to restore the gap 514. Preferably, only one of the electromagnet 502 and the transducer array 510 is energized. That is, when the transducer array 510 is in operation, the electromagnet 502 is not energized, and when the sensor detects that the ultrasound probe 500 is in the acceleration state and the electromagnet 502 is energized, the transducer array 510 is not in operation. Such a configuration can ensure that the transducer array 510 in operation is not affected by a potential magnetic field of the electromagnet 502 (regardless of size), thereby avoiding adverse effects on the quality of ultrasound imaging. Also, while the electromagnet 502 is in operation, the transducer array 501 is not in operation, to maximize the service life of the transducer elements.

Preferably, the ultrasound probe 500 may further include a resilient member 516. The resilient member 516 may be coupled between the transducer module 506 and the base 508, and configured to be compressible to allow relative movement between the transducer module 506 and the base 508. The resilient member 516 may provide cushioning for the approach of the transducer module 506 toward the base 508, and may also provide a support for resetting of the transducer module 506 away from the base 508. Optionally, the resilient member 516 may include at least one spring.

As previously described, the housing 100 may have a dry chamber 102 and a wet chamber 104, the wet chamber 104 being filled with an acoustic liquid, and the transducer module 506 being disposed in the wet chamber 104. Preferably, when the electromagnet 502 is energized to attract the magnetic attraction material 504, a gap between the electromagnet 502 and the magnetic attraction material 504 decreases, and the gap between the transducer array 510 and the contact surface 512 increases, at which time the acoustic liquid in the wet chamber 104 may be more filled into the gap between the transducer array 510 and the contact surface 512.

To facilitate this purpose, as a preferred embodiment, the base of the ultrasound probe may be configured in other shapes. For example, the shape of an upper surface of the base 508 and/or a lower surface of the transducer module 506 may be configured such that the spacing between the transducer module 506 and the base 508 gradually increases from inside to outside in a radial direction. Alternatively, the upper surface of the base 508 may be configured to be upwardly concave and the lower surface of the transducer module 506 may be configured to be downwardly convex. In this way, when the transducer module 506 and the base 508 are proximate to each other, the acoustic liquid is more easily squeezed out of the gap therebetween, thereby increasing a response speed of the system.

In cases where the ultrasound probe is an E4D probe (not shown), the movable transducer module may include all modules needed for 4D ultrasound imaging. Accordingly, the base may be fixedly disposed within the E4D probe and disposed opposite the transducer module.

In cases where the ultrasound probe 500 is a mechanical ultrasound probe, the base 508 may further include a shaft 520, and preferably, the ultrasound probe 500 may further include a driver 522. The driver 522 may drive the scanning head 501 to rotate or oscillate about the shaft 520. The driver 522 may be a motor, and transmit to the base 508 by means of a plurality of gears, the motor continuously rotating alternately in forward and reverse directions, to cause the base to drive the scanning head 501 in an oscillating motion. The manner of oscillation may be any type of driving manner; for example, FIG. 5 shows a case in which two tension cables are present, and the shaft 520 drives movement of the base by means of the tension cables. It is to be noted that the described driving manner is an exemplary illustration. Other driving manners for mechanical ultrasound probes in the art are also permissible.

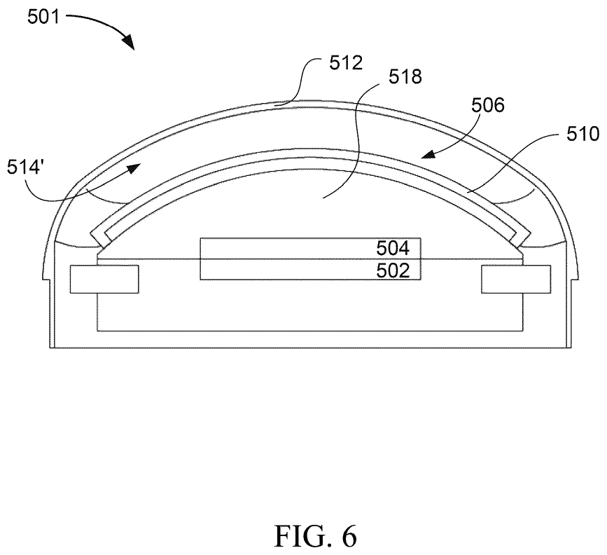

FIG. 6 is a schematic diagram of a scanning head 501 in an attracting state according to one embodiment of the present invention.

In a state shown in FIG. 6, a sensor module 730 detects that the ultrasound probe 500 is in an acceleration state exceeding a threshold. At this time, the electromagnet 502 is energized, the magnetic attraction material 504 is attracted toward the electromagnet 502, and the gap 514 between the transducer array 510 and the contact surface 512 increases to be a gap 514′, so that the fragile transducer module 506 is as far away from the housing as possible, to mitigate external impacts. When this acceleration state ends, the electromagnet 502 is de-energized. The transducer array 510 can be reset to the state shown in FIG. 5 under the action of an elastic force of the resilient member 516. It will be appreciated that the positions of the electromagnet 502 and the magnetic attraction material 504 may be interchanged.

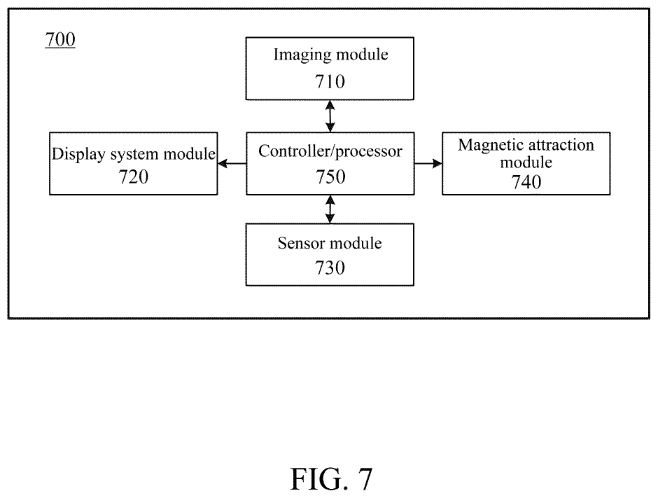

FIG. 7 is a schematic diagram of a medical imaging system 700 according to one embodiment of the present invention. The medical imaging system 700 may include an imaging module 710, a display system module 720, a sensor module 730, a magnetic attraction module 740, and a controller/processor 750. The imaging module 710 may be configured to acquire scan data and generate images. The display system module 720 may be configured to display the images generated based on the scan data to users. The sensor module 730 may be configured to sense an acceleration state of a medical imaging probe. The controller/processor 750 may be configured to determine whether the acceleration state exceeds a threshold, and to enable the magnetic attraction module 740 to attract components within the medical imaging probe when the acceleration state exceeds the threshold, thereby reducing or avoiding impacts on the components within the medical imaging probe. Further, the controller/processor 750 may be configured to enable one of the imaging module 710 and the magnetic attraction module 740 in response to a sensing result. That is, when the magnetic attraction module 740 is enabled, the imaging module 710 is disabled. When the imaging module 710 is enabled, the magnetic attraction module 740 is disabled.

While the present invention has been described with reference to certain implementations, it should be understood by those skilled in the art that various changes may be made and equivalents may be substituted without departing from the scope of the present invention. Furthermore, numerous modifications may be made to adapt particular circumstances or materials to the teachings of the present invention without departing from the scope thereof. Therefore, the present invention is not intended to be limited to the specific embodiments disclosed, but shall encompass all embodiments falling within the scope of the appended claims.

1. A medical imaging probe, comprising:
a housing, a front end of the housing being sealed with a contact surface;
a scanning head, the scanning head being movably disposed within the housing, wherein the scanning head comprises:
a movable transducer module, the movable transducer module comprising a transducer array and a backing, the transducer array being opposite the contact surface, and there being a gap between the transducer array and the contact surface;
a base, the base being coupled to the transducer module and disposed opposite the transducer array;
an electromagnet, the electromagnet being disposed on one of the transducer module and the base; and
a magnetic attraction material, the magnetic attraction material being disposed on the other of the transducer module and the base; and
a sensor, the sensor being configured to sense an acceleration state of the medical imaging probe exceeding a threshold;
wherein the electromagnet and the magnetic attraction material are positioned in such a way that, in response to the acceleration sensor sensing the acceleration state, the electromagnet is energized to attract the magnetic attraction material, causing the transducer module to be proximate to the base, and the gap between the transducer array and the contact surface to increase.

2. The medical imaging probe according to claim 1, further comprising a resilient member, the resilient member being coupled between the transducer module and the base, and configured to be compressible so as to allow relative movement between the base and the transducer module.

3. The medical imaging probe according to claim 2, wherein, when the electromagnet is de-energized, the resilient member resets the transducer module to restore the gap.

4. The medical imaging probe according to claim 2, wherein the resilient member comprises at least one spring coupled between the transducer module and the base.

5. The medical imaging probe according to claim 1, wherein the electromagnet is disposed on the base and the magnetic attraction material is disposed on the transducer module.

6. The medical imaging probe according to claim 5, wherein the magnetic attraction material is disposed on a bottom surface of the transducer module opposite the transducer array.

7. The medical imaging probe according to claim 1, wherein, when the electromagnet is energized to attract the magnetic attraction material, the transducer module is moved towards the base.

8. The medical imaging probe according to claim 1, wherein the electromagnet can be energized only during non-operation periods of the transducer module.

9. The medical imaging probe according to claim 1, wherein the housing comprises a wet chamber filled with an acoustic liquid, and a dry chamber in which the transducer module is disposed,
wherein, when the electromagnet is energized to attract the magnetic attraction material, the acoustic liquid is more filled into the gap between the transducer module and the contact surface.

10. The medical imaging probe according to claim 1, wherein the spacing between the transducer module and the base is configured to gradually increase from inside to outside in at least one radial direction.

11. The medical imaging probe according to claim 1, wherein the base comprises a shaft around which the scanning head can be rotated.

12. The medical imaging probe according to claim 11, further comprising a driver, the driver driving the scanning head to rotate around the shaft.

13. A medical imaging system, comprising:
a memory;
a processor; and
a probe comprising:
a housing, a front end of the housing being sealed with a contact surface;
a scanning head, the scanning head being movably disposed within the housing, wherein the scanning head comprises:
a movable transducer module, the movable transducer module comprising a transducer array and a backing, the transducer array being opposite the contact surface, and there being a gap between the transducer array and the contact surface;
a base, the base being coupled to the transducer module and disposed opposite the transducer array;
an electromagnet, the electromagnet being disposed on one of the transducer module and the base; and
a magnetic attraction material, the magnetic attraction material being disposed on the other of the transducer module and the base; and
a sensor, the sensor being configured to sense an acceleration state of the medical imaging probe exceeding a threshold;
wherein the electromagnet and the magnetic attraction material are positioned in such a way that, in response to the acceleration sensor sensing the acceleration state, the electromagnet is energized to attract the magnetic attraction material, causing the transducer module to be proximate to the base, and the gap between the transducer array and the contact surface to increase.

14. The medical imaging system according to claim 13, further comprising a resilient member, the resilient member being coupled between the transducer module and the base, and configured to be compressible so as to allow relative movement between the base and the transducer module.

15. The medical imaging system according to claim 13, wherein, when the electromagnet is de-energized, the resilient member resets the transducer module to restore the gap.

16. The medical imaging system according to claim 13, wherein the resilient member comprises at least one spring coupled between the transducer module and the base.

17. The medical imaging system according to claim 13, wherein the electromagnet is disposed on the base and the magnetic attraction material is disposed on the transducer module.

18. The medical imaging system according to claim 13, wherein the magnetic attraction material is disposed on a bottom surface of the transducer module opposite the transducer array.

19. The medical imaging system according to claim 13, wherein, when the electromagnet is energized to attract the magnetic attraction material, the transducer module is moved towards the base.

20. The medical imaging system according to claim 13, wherein the electromagnet can be energized only during non-operation periods of the transducer module.
